(19) United States
(12) Patent Application Publication
(10) Pub. No.: US 2009/0240226 A1
(43) Pub. Date: Sep. 24, 2009

(54) Disposable menstrual panties

(76) Inventors: Rudy Lazar Fields, Fontana, CA (US); Ileen Larose Fields, Fontana, CA (US)

Correspondence Address: Rudy Fields, 5029 LaSarre Dr., Fontana, CA, 92336, US

(21) Appl. No.: 12/077,515

(22) Filed: Mar. 19, 2008

(51) Int. Cl. A61F 13/15

(52) U.S. Cl. 604/385.01; 604/396

(57) ABSTRACT

A disposable menstrual panty comprising of a garment having a front portion, a rear portion, a crotch portion having two leg openings, a right leg opening and a left leg opening and a waist portion as to be worn about the lower torso of a women before during and after menstrual cycle. A sanitary napkin is stitched and permanently bonded with an adhesive material within the entire crotch portion that will not slide or curl up around the edges ensuring stability. Garment also has side attachments that join together front portions and rear portion of panty at both left and right sides of panty using velcro attachments or alternate adhesive tape fasteners or tie string.

### FIELD OF INVENTION

This present invention relates to under garments in generally and more specifically a disposable panty for women for the protection of pre/post and normal menstrual needs.

### BACKGROUND OF INVENTION

### Summary of Prior Art

Researches into disposable panties have shown to be enormous. Searches revealed several types of disposable panties including those with inside crotch pockets to those designed to allow for the placement of sanitary napkins of various sizes. Other disposable panties have also been found to resemble that of a pamper or diaper. Some have been found to have the pad combined to the panty. These designs though unique in their own right still pose inconsistencies to women's needs, comforts and accessibility. Although the prior arts may provide for some comfort they unfortunately lack the ease for placement and removal of disposable panty. Women in today's society are not limited to dresses alone. Research had shown that more women have now changed their styles from dresses to pants. Thus making the prior art of disposable panties complicated to remove while wearing pants.

### SUMMARY OF INVENTION

The present invention provides protection from liquid leakage of menstrual periods. The present invention makes up for the inconsistencies of prior arts and provides comfort and ease of a new desirable design.

Accordingly the garment consists of a disposable panty which in itself is combined with a sanitary napkin being permanently stitched and bonded. The sanitary napkin itself has a polyethylene backing that provides barrier protection needed to prevent liquid strike through.

The napkin is permanently secured to the bottom of the inner crotch portion of panty with an adhesive material which will prevent napkin from sliding and shifting during wear. Sanitary napkin is stitched within the seems along the entire crotch portion of panty to prevent edges from curling upward during wear and allows for a more comfortable fit for wearer.

Furthermore, unlike the prior art, the object is to provide a menstrual panty that is simple and easy to use. Therefore said garment comes with side attachments, either Velcro, adhesive or tie-string for easy placement and removal.

Unlike the prior art, disposable panty can be worn with dresses, skirts, pants and pant type garments.

Research has indicated that 9 out of 10 women wear pants or pant type garments 90% of the time. Therefore, having a disposable panty that will allow for easy removal with pants will be highly desirable for most women.

Knowing that fashion trends are important to most women, said garment is very inexpensive and comes in fashionable bikini, low rise, hip hugger and brief type designs that can vary in color and size.

Disposable panty is manufactured with materials made with nylon, cotton and spandex. To prevent accidental soiling of clothing, disposable panty also supports several usages. Disposable panties can serve as pre-menstrual wear which would be worn just prior to regular cycle to prevent accidental soiling onto clothing. Disposable panty can also serve as post-menstrual wear which would be worn during days following regular menstrual cycle to prevent accidental soiling onto clothing.

In both cases of pre and post menstrual protection, the combined pad included in disposable panty would be in the form of a panty liner, thus giving protection for possible soiling of clothing which often occurs in most women.

### BRIEF DESCRIPTION OF DRAWINGS

The present invention will be fully understood and will become more apparent when references are made to the following description.

FIG. 1 is a perspective view of full-sized disposable menstrual panty of present invention.

FIG. 2 is and exploded elevated view of panty garment of present invention in a pre-assembled flat configuration.

FIGS. 3a & b are side views showing waist velcro attachment portion of said panty in an open position. This attachment secures together side ends of front and back on both right and left side of panty.

FIGS. 4a & b are side views showing alternate adhesive attachments for both left and right sides of panty in a closed position. Adhesive attachments being a free end tape fastener coated with pressure sensitive adhesive along with a receiving end, target tape strip.

FIGS. 5a & b are side views showing alternate attachments, tie-string on both right and left sides for securing panty garment together.

FIG. 6 front elevated view of the disposable panty being worn by wearer.

### DETAILED DESCRIPTION OF PREFERRED EMBODIMENT

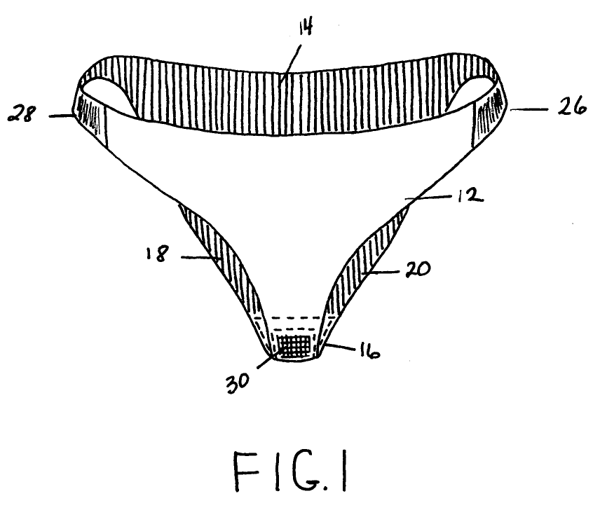

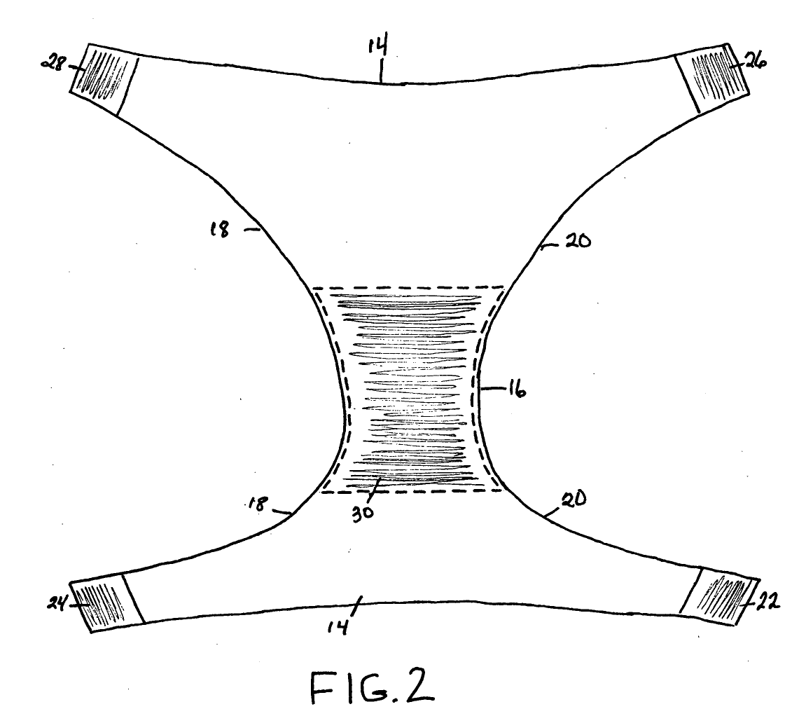

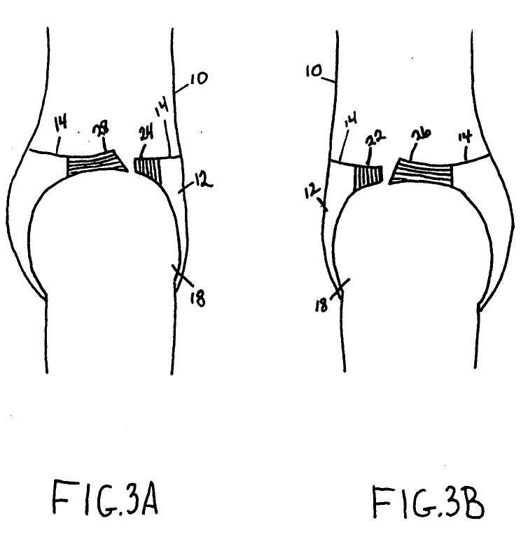

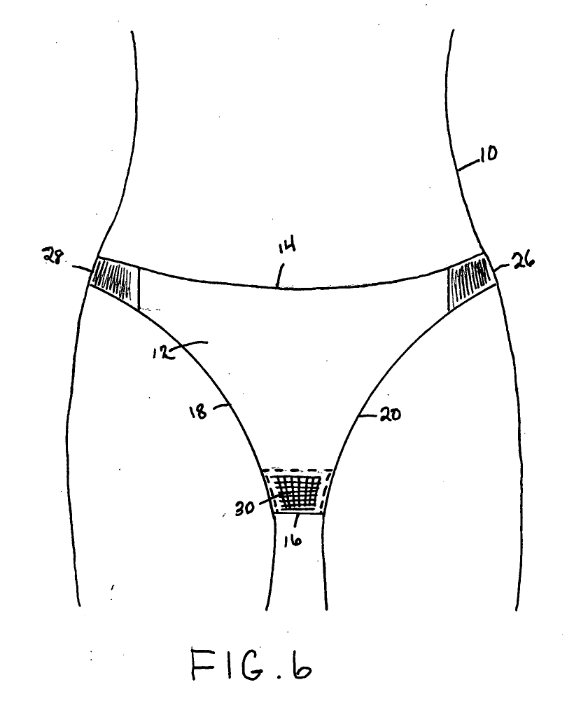

Referring now descriptively to the drawings. FIG. 1-6 illustrates a disposable panty 12, worn by a woman wearer 10. The disposable panty 12, configured with a waist portion 14, a crotch portion 16, having a right leg opening 18, and a left leg opening 20. Disposable panty 12, has side velcro attachments 22 and 24, receiving ends, whereby securing opposite side velcro attachments 26 and 28, target strip ends of panty 12, together to form a bikini type garment.

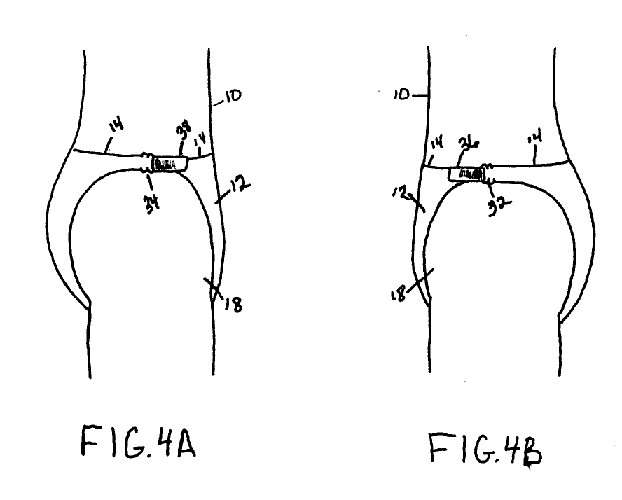

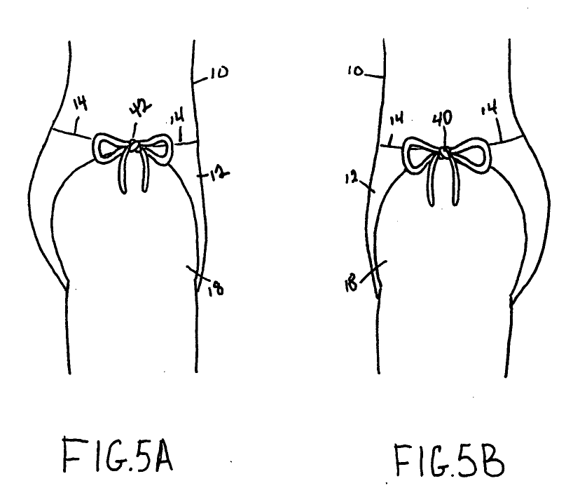

Disposable panty 12 comes combined with a stitched and bonded in sanitary napkin 30, for wear during normal menstrual periods. Disposable panty 12 also comes combined with an alternate stitched and bonded in panty liner 30, for wear before and after normal menstrual periods. FIGS. 4a & b denotes other attachment means which can also be used such as, adhesive tape fasteners; left and right 32 & 34, receiving ends and target tape strips and 36 and 38. FIGS. 5a & b denotes alternate attachments which can also be used such as tie-string left and right, 40 & 42.

### REFERENCE NUMERALS

10 Women Wearer
12 Disposable panty
14 Waist opening
16 Crotch portion
18 Right leg opening
20 Left leg opening
22 Side attachments (lft) Velcro (rec.end)
24 Side attachments (rt) Velcro (rec.end)
26 Side attachments (lft) Velcro (free end fasteners)
28 Side attachments (rt) Velcro (free end fasteners)
30 Stitched/bonded in sanitary napkin/panty liner
32 Alternate side attachments (lft) adhesive tape fasteners
34 Alternate side attachments (rt) adhesive tape fasteners
36 Target tape strip, receiving end (lft)
38 Target tape strip, receiving end (rt)
40 Alternate side attachments (lft) tie-string
42 Alternate side attachments (rt) tie-string

What is claimed is:

1. A disposable menstrual panty comprising:
a) a garment configured with a front portion, a rear portion, and crotch area having two leg openings and a waist opening.
b) an absorbent padded barrier consisting of a sanitary napkin being stitched and permanently bonded to the inner portion of the crotch area of disposable panty, extending across the width of the crotch area and positioned from the front end of crotch to the rear end of crotch.
c) sanitary napkin is stitched within the seems of entire crotch portion of said garment which secures napkin to the panty and prevents edges from curling upward during wear.
d) a permanent bonding adhesive is applied to the bottom portion of sanitary napkin to prevent napkin from sliding and shifting about the crotch portion during wear of said garment.

2. A disposable menstrual panty containing all limitations of claim 1 and including additional limitations wherein disposable panty being detachable and attachable at the right and left sides of hip areas.
a) attachments on both right waist sides and left waist sides to allow for fastening and unfastening of said garment.
b) attachments include, Velcro or adhesive tape or tie string both sides of said garment.

3. A disposable menstrual panty containing all limitations of claim 1 and including additional limitations wherein a panty liner which provides protection before, during and after menstrual cycle may also be stitched within the seems of the entire crotch portion of said garment which secures the panty liner to the panty and prevents edges from curling upward during wear.

4. A disposable menstrual panty containing all limitations of claim 2 and including additional limitations wherein front portions and rear portions of said panty are joined together at both sides to form a bikini, low rise, hip hugger or brief type disposable menstrual panty.
